(19) United States
(12) Patent Application Publication
(10) Pub. No.: US 2017/0333554 A1
(43) Pub. Date: Nov. 23, 2017

(54) DOSING REGIMEN

(71) Applicant: IMCLONE, LLC, Indianapolis, IN (US)

(72) Inventors: John Robert BALDWIN, Martinsville, IN (US); Ilaria CONTI, Carmel, IN (US); Damien Michel CRONIER, Twickenham (GB); Robert Leslie ILARIA, Jr., Zionsville, IN (US); Zhi Lin MO, Carmel, IN (US)

(73) Assignee: IMCLONE, LLC, Indianapolis, IN (US)

(21) Appl. No.: 15/590,042

(22) Filed: May 9, 2017

Related U.S. Application Data
(60) Provisional application No. 62/338,609, filed on May 19, 2016.

(51) Int. Cl. A61K 39/395; C07K 16/18; C07K 14/47

(52) U.S. Cl. CPC A61K 39/395; C07K 14/4748; C07K 16/18

(57) ABSTRACT

The invention provides for a dosing regimen for human antibodies that bind to human platelet-derived growth factor receptor alpha (PDGFR alpha), preferably olaratumab.

This invention is directed to the fields of immunology and cancer treatment. More specifically, the present invention is directed to the dosing of olaratumab as a medicament for the treatment of cancer.

The present invention seeks to provide a method of administering olaratumab in response to a clinically unmet need for patients who fail to respond to the current olaratumab dosing regimens. In this regard, the olaratumab dosing regimen of the present invention provides an unmet need for patients who fail the standard dosing regimens of olaratumab. Few to no therapeutic approaches are currently available to these patients, including but not limited to, patients with advanced soft tissue sarcoma (STS). Accordingly, the present invention provides a significant unmet need in a patient population that lacks clinical options.

A novel and inventive dosing strategy of olaratumab for the treatment of cancer is herein presented. Olaratumab, IMC-3G3 (U.S. Pat. Nos. 8,128,929 and 8,574,578), is a recombinant human monoclonal antibody that specifically targets the human platelet-derived growth factor receptor alpha (PDGFRα or PDGFR alpha). These patents disclose the treatment of a variety of neoplastic diseases, including soft tissue sarcomas, with PDGFRα antibodies, including IMC-3G3. Dosing for olaratumab is referenced therein. “An exemplary, non-limiting range for a therapeutically effective amount of [olaratumab] is “0.1-50 mg/kg, more preferably 3-35 mg/kg, and more preferably 5-20 mg/kg” can be found in WO 2016/003789. Olaratumab dosing schedules of 15 mg/kg intravenously on day 1 and day 8 for up to 8 cycles can be found at (NCT01185964 as available on www.clinicaltrials.gov, first received on Aug. 19, 2010) and WO 2016/003789. WO 2016/003789 notes that “dosages normally are given on a 21-day cycle normally on days one and eight and each dose falls within the range of about 10 mg/kg to about 18 mg/kg, preferably about 13.5 mg/kg to about 16.5 mg/kg, and most preferably about 15 mg/kg.” The use of a loading dose for olaratumab was not previously contemplated, less the specific dosing regimen as herein set forth in some aspects of the present invention.

Drug development is unpredictable. It is common for new molecules to fail in preclinical and/or clinical stages, often for little understood reasons. Dosing is an added complexity that drives further unpredictability. Not all drug dosing regimens are equally active in all patient populations due to various factors, including but not limited to, body weight, performance status, number of prior systemic therapies, genetics, and histological tumor type. Toxicity issues introduce additional complexities. Efficacy and toxicity must be balanced. Additionally, it is not always possible to predict which patients will achieve therapeutic serum levels fast enough to receive benefit before disease progression. Administering a loading dose seeks to provide possible remediation of early progression in the previously non-responding subpopulation without resulting in toxicological barriers for that population or the previously responding population.

The results from study number NCT01185964 (as available on www.clinicaltrials.gov, first received on Aug. 19, 2010) (hereinafter “Study”), as further illustrated in WO2016/003789, demonstrate a clinically significant and unexpected benefit for olaratumab in soft tissue sarcoma patients. Olaratumab provides a significant benefit to patients, especially as measured through progression-free survival (PFS) and overall survival (OS) of patients, as seen in the Study and WO2016/003789 using 15 mg/kg. Despite these tremendously significant results obtained with the 15 mg/kg regimen, a subset of patients fails to benefit from olaratumab, leaving them with limited to no other beneficial treatment options. Upon further investigation, patient response to olaratumab dosed as described in WO2016/003789, was found to vary based on the patient serum exposure to olaratumab. Accordingly, it is believed that some patients do not receive the full benefit of olaratumab because these patients do not reach the required therapeutic serum levels of olaratumab fast enough to avoid early disease progression. Based on the PK/PD analysis presented herein, patients in the lowest exposure quartile in the study showed no improvement in OS. These, patients seemed to fail treatment and progress during the first 2-3 cycles of therapy. Exposure-response modelling and matched case-control analysis of OS and PFS per exposure quartiles based on the trough olaratumab serum concentration at the end of Cycle 1 (C_{min,1}) showed these patients progressed prior to reaching a therapeutic window with therapeutic olaratumab serum levels. Nothing in the preclinical or clinical studies with olaratumab suggested using, or the necessity of using, a loading dose to reach the therapeutic window more quickly. This highlights the complexity and difficulty involved in establishing dosing regimens that are able to achieve maximal benefit to all patient populations. Although loading doses are not uncommon, use of a loading dose is also not common among recently approved oncology therapeutics.

Further, there was nothing in the olaratumab data that would suggest the need for the dosing regimen of the present invention. Additionally, there is nothing that would suggest the specific dosing regimen as set forth in some aspects of the present invention. There is nothing that would suggest or provide insight into the fact that the therapeutic window could be achieved by the present invention, less the specific regimen set forth herein. Thus, for olaratumab, the use of the loading dose to achieve therapeutic olaratumab serum levels more quickly is unanticipated.

The present invention is being studied in a randomized Phase 3 trial (Study Number NCT02451943, as available on www.clinicaltrials.gov, first received on May 20, 2015 (hereinafter “Phase 3 Study”). The patients in this Phase 3 Study, as disclosed therein, are given a loading dose of 20 mg/kg on day 1 and day 8 of cycle 1 followed by 15 mg/kg on day 1 and day 8 of every subsequent cycle.

The clinical results from the analysis of the Study (Phase 2 NCT01185964 study) were surprising because the data illustrate a 6.6 month PFS and, perhaps even more importantly, a 26.5 month OS, with a hazard ratio (HR) of 0.672 (95% CI, 0.442-1.021; p=0.0615) for PFS and a HR of 0.46 (95% CI, 0.30-0.71; p=0.0003) for OS. The observed significant improvement over the current standard of care in terms of a lower HR and a larger improvement in median survival time were highly unexpected. In spite of these unexpected and tremendous results, upon review of the final data, it was found that olaratumab failed to achieve a clinically significant response in a percentage of the study population. The present invention seeks to provide a dosing regimen that provides a benefit to this previously unresponsive patient population to meet this important clinically unmet need.

According to the first aspect of the invention, there is provided a method of administering an effective amount of olaratumab to a patient in need thereof comprising administering a loading dose of olaratumab followed by standard doses of olaratumab. In a preferred aspect of the invention, the loading dose of olaratumab comprises 20 mg/kg of olaratumab. In another aspect of the invention, the 20 mg/kg of olaratumab is administered on day 1 and day 8 of a 21 day cycle. In a further aspect of the invention, the 20 mg/kg of olaratumab is administered in the first cycle. In a preferred aspect of the invention, standard dose comprises 15 mg/kg of olaratumab. In another aspect of the invention, the 15 mg/kg of olaratumab is administered in the subsequent cycles. In a further aspect of the invention, the 15 mg/kg of olaratumab is administered on day 1 and day 8 of a 21 day cycle. In another aspect of the invention, the patient has or is at risk of developing early progression. In yet another aspect of the invention, the patient has soft tissue sarcoma.

In another aspect of the invention, there is provided a method of administering an effective amount of olaratumab to a patient in need thereof comprising administering cycles of the olaratumab wherein a loading dose of 20 mg/kg of olaratumab is administered in the first cycle and a standard dose of 15 mg/kg of olaratumab is administered in the subsequent cycles. In an aspect of the invention, the loading dose of 20 mg/kg of olaratumab is administered in the first 21 day cycle and the standard dose of 15 mg/kg of olaratumab is administered in the subsequent 21 day cycles. In another aspect of the invention, the loading dose of 20 mg/kg of olaratumab is administered on day 1 and day 8 of the first 21 day cycle and the standard dose of 15 mg/kg of olaratumab is administered on day 1 and day 8 of the subsequent 21 day cycles. In a further aspect of the invention, the patient has or is at risk of developing early progression. In yet another aspect of the invention, the patient has soft tissue sarcoma.

Another aspect of the present invention is a therapeutic regimen for olaratumab in the treatment of cancer comprising 21 day cycles wherein a loading dose of 20 mg/kg of olaratumab is administered on day 1 and day 8 of the first 21 day cycle and 15 mg/kg of olaratumab is administered to a patient in need thereof on day 1 and day 8 of the subsequent 21 day cycles. In another aspect, the patient has or is at risk of developing early progression. In a further aspect, the cancer is soft tissue sarcoma.

Yet another aspect of the present invention is a method reducing the risk of disease progression of a patient having soft tissue sarcoma comprising administering a loading dose of olaratumab followed by standard doses of olaratumab. In a preferred aspect, the loading dose of olaratumab comprises 20 mg/kg of olaratumab. In another aspect, the 20 mg/kg of olaratumab is administered on day 1 and day 8 of a 21 day cycle. In yet another aspect, the 20 mg/kg of olaratumab is administered in the first cycle. In a preferred aspect, the standard dose comprises 15 mg/kg of olaratumab. In another aspect, 15 mg/kg of olaratumab is administered in subsequent cycles. In yet another aspect, the 15 mg/kg of olaratumab is administered on day 1 and day 8 of a 21 day cycle.

In yet another aspect of the present invention, there is provided a method of treating soft tissue sarcoma in a patient in need there of comprising administering a loading dose of olaratumab followed by standard doses of olaratumab. In a preferred aspect, the loading dose of olaratumab comprises 20 mg/kg of olaratumab. In another aspect, the 20 mg/kg of olaratumab is administered on day 1 and day 8 of a 21 day cycle. In another aspect, the 20 mg/kg of olaratumab is administered in the first cycle. In another preferred aspect, the standard dose comprises 15 mg/kg of olaratumab. In a further aspect, the 15 mg/kg of olaratumab is administered in the subsequent cycles. In another aspect, the 15 mg/kg of olaratumab is administered on day 1 and day 8 of the 21 day cycle. In yet another aspect, the patient has or is at risk of developing early progression.

### DESCRIPTION OF THE FIGURE

FIG. 1 depicts the predicted effect of olaratumab on the hazard ratio for progression-free survival. Progression-free survival as predicted by Cmin1-based model (left panel) and the C_{avg}-based model (right panel). The solid red lines describe the change in HR as a function of Cₘᵢₙ₁ and C_{avg}; the grey histograms describe the distribution of olaratumab Cₘᵢₙ₁ and C_{avg} in the “Study” experimental arm together with their quartiles (dashed blue lines); the green dashed lines indicate ECₘᵢₙ₁50 and EC_{avg}50. Abbreviations: C_{avg}=average concentration over patient's entire treatment; Cₘᵢₙ₁=trough serum concentration at the end of Cycle 1; EC_{avg}50=olaratumab C_{avg} yielding a 50% decrease in the baseline hazard; ECₘᵢₙ₁50=olaratumab Cₘᵢₙ₁ yielding a 50% decrease in the baseline hazard; HR=hazard ratio.

The invention provides for olaratumab in various aspects disclosed herein. As used herein, the term “olaratumab”—also known as IMC-3G3, CAS registry number 1024603-93-7—refers to an anti-PDGFRα antibody comprising: two heavy chains, each of whose amino acid sequence is that given in SEQ ID NO: 8, and two light chains, each of whose amino acid sequence is that given in SEQ ID NO: 16 as set forth and claimed in U.S. Pat. No. 8,128,929.

Olaratumab is a recombinant human monoclonal antibody of the IgG₁ isotype that specifically targets human PDGFRα. The antibody possesses high-affinity binding for PDGFRα and blocks platelet-derived growth factor-AA (PDGF-AA), -BB, and -CC ligands from binding to the receptor. As a result, olaratumab inhibits ligand-induced receptor autophosphorylation and phosphorylation of the downstream signaling molecules protein kinase B (Akt) and mitogen-activated protein kinase (MAPK). Olaratumab inhibits the proliferation and growth of a variety of human tumor cell lines.

The terms “platelet-derived growth factor receptor alpha,” “platelet-derived growth factor receptor α,” “PDGFR alpha,” “PDGFRα,” “PDGFRα,” “PDGF alpha receptor,” and “PDGFα receptor” are used interchangeably herein, unless otherwise indicated, and are intended to refer to the human type III receptor tyrosine kinase, as well as functionally active, mutated forms thereof, that bind human platelet-derived growth factor. Specific examples of PDGFRα include, e.g., a human polypeptide encoded by the nucleotide sequence provided in GenBank® accession no. NM_006206.4, or the human protein encoded by the polypeptide sequence provided in GenBank® accession no. NP_006197.1.

PDGFRα is a receptor tyrosine kinase that can be activated by platelet-derived growth factor (PDGF)-AA, -AB, -BB, and -CC. These growth factors are dimeric molecules composed of disulfide-linked polypeptide chains that bind to two receptors simultaneously and induce receptor dimerization, autophosphorylation, and downstream intracellular signaling. PDGFRα is expressed in many mesenchymal structures; thus, PDGFRα plays a critical role during early and later stages of development.

The term “soft tissue sarcoma” or “STS,” as used herein, is a malignancy that originates in soft or connective tissue like fat, muscle, nerves, fibrous tissues, blood vessels, or deep skin tissues; all of mesenchymal origin. More than 50 histological subtypes of STS have been identified. “Soft tissue sarcoma” or “STS” include but are not limited to: alveolar soft part sarcoma, chondrosarcoma, clear cell sarcoma, endometrial stromal sarcoma, epithelial sarcoma, epithelioid, extraskeletal myxoid chondrosarcoma, fibromyxoid sarcoma, fibrosarcoma, fibrosarcomatous transformation in a recurrent dermatofibrosarcoma, hemangiopericytoma, high grade undifferentiated sarcoma, leiomyosarcoma, liposarcoma, malignant fibrous histiocytoma, malignant glomus tumor, malignant peripheral nerve sheath tumor, malignant solitary fibrous tumor, malignant spindle cell sarcoma, malignant spindle cell tumor with rhabdoid features, myxofibrosarcoma, myxoid chondrosarcoma, myxoid liposarcoma, myxoid sarcoma, neurofibrosarcoma, pleomorphic sarcoma (including: high grade spindle and pleomorphic sarcoma, smooth muscle tumor, solitary fibrous tumor, spindle cell sarcoma, undifferentiated), rhabdomyosarcoma, synovial sarcoma, undifferentiated sarcoma, and undifferentiated uterine sarcoma. Due to the nature of the disease state and origin of the tumor, a patient may be diagnosed as one or more of the subtypes including the exemplary subtypes disclosed herein.

Treatment options for STS, including subtypes exemplified in the Study, continue to be limited. Despite the clinically meaningful and highly statistically significant results demonstrated in the Study (NCT01185964), there remains a subset of patients in which olaratumab fails to demonstrate efficacy. This subset includes patients who had subtherapeutic drug levels before reaching steady state drug levels. The present invention allows these patients to achieve therapeutic steady state levels much earlier in their treatment course. Current therapies have shown little to no clinical benefit for these patients. There is a high clinical unmet need for treatment options including dosing regimens for this subset of patients that provide a survival benefit; the present invention seeks to provide such.

As used herein, the term “advanced” STS refers to any of the following criteria including but not limited to: (1) unresectable, (2) metastatic, (3) histologically or cytologically confirmed documented disease progression, or (4) not amenable to treatment with surgery or radiotherapy.

As used herein, “about” means±5%.

As used herein, “doxorubicin” is a cytotoxic antibiotic of the anthracyline family of compounds. Its cytotoxic effects are believed to stem from intercalation with DNA nucleotides leading to inactivation of topoisomerase II DNA repair, and generation of free radicals leading to lipid peroxidation and cell membrane damage. Investigations of cellular exposure to doxorubicin have shown morphological changes associated with apoptosis.

As used herein, “dexrazoxane” or “dexrazoxane hydrochloride” is a cardioprotective agent. It is commonly used to protect the heart against the cardiotoxic side effects of anthracyclines, such as daunorubicin or doxorubicin.

As used herein, the terms “treating,” “treat,” or “treatment” refers to restraining, slowing, lessening, reducing, or reversing the progression or severity of an existing symptom, disorder, condition, or disease or ameliorating clinical symptoms of a condition. Beneficial or desired clinical results include, but are not limited to, alleviation of symptoms, diminishment of the extent of a disease or disorder, stabilization of a disease or disorder (i.e., where the disease or disorder does not worsen), delay or slowing of the progression of a disease or disorder, amelioration or palliation of the disease or disorder, and remission (whether partial or total) of the disease or disorder, whether detectable or undetectable. Treatment can also mean prolonging survival as compared to expected survival if not receiving treatment. Those in need of treatment include those already with the disease. In one embodiment, the present invention can be used as a medicament.

As used herein, the terms “cancer” and “cancerous” refer to or describe the physiological condition in mammals that is typically characterized by unregulated cell growth. Included in this definition are benign and malignant cancers.

Although human antibodies of the invention are particularly useful for administration to humans, they can be administered to other mammals as well. Accordingly, as used herein, the term “patient” refers to a mammal, preferably a human. The term mammal as used herein is intended to include, but is not limited to, humans, laboratory animals, domestic pets and farm animals.

A “therapeutically effective amount,” or “effective amount” as used herein, refers to an amount effective, at dosages and for periods of time necessary, to achieve the desired therapeutic result. An effective amount can be readily determined by the attending diagnostician, as one skilled in the art, by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the target site; the degree of or involvement or the severity of the disease or disorder; the response of the individual patient; the particular compound administered; the mode of administration; the bioavailability characteristics of the preparation administered; the dose regimen selected; the use of concomitant medication; other medications administered; and other relevant circumstances. A therapeutically effective amount is also one in which any toxic or detrimental effects of the antibody or antibody portion are outweighed by the therapeutically beneficial effects.

As used herein, the term “loading dose” means a larger initial dose or doses that are administered before a standard dose to hasten the increase in serum drug levels.

As used herein, the terms “effective response” of a patient or a patient's “responsiveness” to treatment with the agents, or “therapeutic effect” refers to the clinical or therapeutic benefit(s) imparted to a patient upon administration. As used herein, an “unexpected therapeutic effect” of the treatment of the invention is the ability to produce marked anti-cancer effects in a patient without causing significant toxicities or adverse effects, so that the patient benefits from the treatment overall. The efficacy, i.e., therapeutic effect(s), of the treatment of the invention can be measured by various endpoints commonly used in evaluating cancer treatments, include any one or more including, but not limited to: extending survival (including OS and PFS); resulting in an objective response (including a CR or a PR); tumor regression, tumor weight or size shrinkage; longer time to disease progression; increased duration of survival; longer PFS; improved OS rate; increased duration of response; and improved quality of life and/or improving signs or symptoms of cancer, etc. Novel approaches to determine efficacy, i.e., therapeutic effect(s), of the present invention can be optionally employed, including, for example, measurement of plasma or urinary markers of angiogenesis and measurement of response through radiological imaging.

As used herein, the term “therapeutic window” means the range of dose or concentration in a patient that gives the treatment the ability to produce marked anti-cancer effects without causing significant toxicities or adverse effects, so that the patient benefits from the treatment overall.

As used herein, the term “therapeutic serum levels” or “therapeutic drug levels” or “therapeutic drug concentration” means the range of drug serum levels that fall within the aforementioned therapeutic window. Serum levels can be measured by an enzyme-linked immunosorbent assay; a test that uses antibodies and color change to identify and quantify a substance.

As used herein, the term “steady state” refers to the situation where the overall intake of a drug is essentially in dynamic equilibrium with its elimination. Practically, “steady state exposure” means the range of drug serum concentration achieved once steady state is reached.

As used herein, the term “disease progression” or “progressive disease” (PD), used interchangeably herein, refers to at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. The appearance of one or more new lesions is also considered progression.

As used herein, the term “partial response,” (PR) refers to at least a 30% decrease in the sum of diameters of target lesions, taking as reference the baseline sum diameters.

As used herein, the term, “complete response” (CR) refers to the disappearance of all target lesions. Any pathological lymph nodes (whether target or non-target) must have reduction in short axis to <10 mm.

As used herein, the term “stable disease” (SD) refers to a tumor diameter that does not shrink sufficiently to qualify for PR or sufficient increase to qualify for PD, taking as reference the smallest sum diameters while on Study.

As used herein, the term “objective response” (OR) refers to a measurable response, including CR or PR.

As used herein, the term, “overall survival” (OS) refers to the patient remaining alive for a defined period of time, such as 1 year, 5 years, etc. from the time of diagnosis or treatment. In a preferred aspect of the invention, for the Study, overall survival is defined as the time from the date of randomization in the Study to the date of death from any cause; if the patient is alive at the end of the follow-up period or is lost to follow-up, OS will be censored on the last date the patient is known to be alive.

As used herein, the term, “progression-free survival” (PFS) refers to the patient remaining alive without the cancer progressing or getting worse. In a preferred aspect of the invention, PFS is defined as the time from randomization in the Study until the first radiographic documentation of objective progression as defined by RECIST (Version 1.1), or death from any cause. Patients who die without a reported prior progression will be considered to have progressed on the day of their death. Patients who did not progress or are lost to follow-up will be censored at the day of their last radiographic tumor assessment.

As used herein, the term “extending survival” or “prolonged survival” which are used interchangeably herein, refers to increasing OS or PFS in a treated patient relative to i) an untreated patient, ii) a patient treated with less than all of the anti-tumor agents in a particular combination therapy, or iii) a control treatment protocol. Survival is monitored for at least about one month, at least about two months, at least about four months, at least about six months, at least about nine months, or at least about 1 year, or at least about 2 years, or at least about 3 years, or at least about 4 years, or at least about 5 years, or at least about 10 years, etc., following the initiation of treatment or following the initial diagnosis of cancer.

Olaratumab can be administered in combination with one or more other anti-cancer treatments, including, but not limited to, an anti-angiogenesis agent, a chemotherapeutic agent, and an anti-neoplastic agent. Any suitable anti-cancer agent can be used, such as a chemotherapeutic agent, radiation, antibody or combinations thereof. Anti-cancer agents include but are not limited to anti-neoplastic agents, antibodies, adjuvants, and prodrugs. Olaratumab can be administered with antibodies and/or small molecules that inhibit and/or modulate other cell surface receptors involved in tumor growth or angiogenesis or extracellular matrix proteins/factors or epithelial/mesenchymal transitions. It can also be administered in combination with one or more suitable adjuvants, such as, for example, cytokines or other immune stimulators, such as, but not limited to, chemokine, tumor-associated antigens, and peptides. In a preferred aspect of the invention, the anti-cancer agent is doxorubicin.

In the present invention, any suitable method or route can be used to administer olaratumab, and optionally, to co-administer anti-neoplastic agents and/or antagonists of other receptors. Olaratumab can be administered before, during, substantially simultaneous with, or after commencing therapy with another agent. Routes of administration include, for example, oral, intravenous, intraperitoneal, subcutaneous, or intramuscular administration; intravenous administration is the preferred route. It should be emphasized, however, that the present invention is not limited to any particular method or route of administration.

Olaratumab, where used in a mammal for the purpose of treatment, are preferably formulated as pharmaceutical compositions. Such pharmaceutical compositions and processes for preparing the same are well known in the art. See, e.g. Remington: Practice of The Science and Pharmacy, Gennaro, 19th Edition, Mack Publishing Company 1995. Olaratumab is preferably formulated as pharmaceutical compositions administered by any route that makes the compound bioavailable. The route of administration may be varied in any way, limited by the physical properties of the drugs and the convenience of the patient and the caregiver. Preferably, olaratumab compositions are for parenteral administration, such as intravenous (i.v.) administration. Such pharmaceutical compositions and processes for preparing same are well known in the art. (See e.g., id.). The route of administration may be varied in any way, limited by the physical properties of the drugs and the convenience of the patient and the caregiver.

Olaratumab may be administered simultaneously, separately, or sequentially with another anti-cancer regimen. As used herein, the phrase “in combination with” refers to the administration of olaratumab and another anti-cancer regimen simultaneously, sequentially in any order, or any combination thereof. The two compounds may be administered in separate pharmaceutical compositions. Olaratumab can be administered prior to, at the same time as, or subsequent to administration of another anti-cancer regimen or in some combination thereof.

The following non-limiting examples and assays further illustrate the present invention and the unexpected benefits thereof.

The following assays, analyses, and results illustrate the unexpected improvement of the dosing regimen for olaratumab as modelling, and pharmacokinetics and exposure response analysis.

The following assays and clinical study designs further illustrate the invention, but should not be construed to limit the scope of the invention in any way.

Olaratumab can be made, for example, according to the disclosure in U.S. Pat. No. 8,128,929.

A pharmacokinetics (PK) and exposure-response analysis was performed on the clinical Phase 2 data resulting from the Study (“A Randomized Phase 2 Study Evaluating the Efficacy of Doxorubicin With or Without Olaratumab in the Treatment of Soft Tissue Sarcoma”, NCT01185964; the Study and resulting interim data were disclosed in WO2016/003789). Olaratumab demonstrated overall survival across all subtypes in the Study however, not all patient populations saw this benefit. The PK and exposure-response analysis explores the possible change in dosing strategy needed for this patient population.

“The Study” and its Final Results (“Randomized Phase 2 Study Evaluating the Efficacy of Doxorubicin with or without Olaratumab in the Treatment of Soft Tissue Sarcoma”, NCT01185964)

### Study Design:

The Study is an open-label, multicenter, Phase 2 trial in which patients with advanced STS not amenable to treatment with surgery or radiotherapy are treated with olaratumab in combination with doxorubicin or doxorubicin alone.

Enrolled patients who meet all eligibility criteria are randomized 1:1 to one of two treatment arms, Arm A or Arm B. Arm A patients receive olaratumab in combination with doxorubicin (hereinafter the “Combination Arm”) and Arm B patients receive doxorubicin alone (hereinafter the “Doxorubicin Arm”). Randomization of this Study is performed according to a dynamic randomization algorithm using 4 pre-defined risk factors: PDGFRα expression (positive vs. negative), number of previous lines of treatment (0 vs. 1+lines), histological tumor type (i.e., leiomyosarcoma vs. synovial sarcoma vs. other tumor type), and ECOG performance status (0-1 vs. 2). This dynamic randomization procedure is used to minimize imbalance between treatment groups. To determine tumor type and PDGFRα expression in patients, a tumor sample from either a biopsy or previously archived tumor tissue sample is sufficient. Biopsy is required to be performed within 21 days prior to randomization; archived tissue sample must also be available within this timeframe.

Combination Arm:

66 patients randomized in the Combination Arm receive 15 mg/kg of olaratumab on Days 1 and 8 of each 21-day treatment cycle, in combination with 75 mg/m² doxorubicin administered on Day 1 of each cycle. (Note: if premedication is required prior to the first doxorubicin infusion, this is to be done after the completion of olaratumab infusion, not before the olaratumab infusion. This premedication may be administered within the hour that follows completion of the olaratumab infusion.) The actual (not planned) administration of each doxorubicin infusion defines Day 1 of each 21-day treatment cycle. The combination treatment just described is to continue for up to 8 cycles at the investigator's discretion. During Cycles 5 through 8, patients being treated with doxorubicin may also be administered 750 mg/m² dexrazoxane on Day 1 of each cycle, (at the investigator's discretion) prior to doxorubicin on Day 1 of each 21-day cycle. If the dose of doxorubicin is reduced at any point in the Study, the dose of dexrazoxane is to be reduced accordingly, maintaining a dosage ratio of 10:1. For example, if doxorubicin is reduced to 60 mg/m², then dexrazoxane is to be reduced to 600 mg/m². Doxorubicin may be administered within the hour that follows completion of the olaratumab infusion. In the absence of PD or other withdrawal criteria, patients in the Combination Arm who have completed eight cycles of therapy are to receive subsequent olaratumab monotherapy, administered on Days 1 and 8 of each 21-day cycle. Furthermore, if doxorubicin is discontinued (e.g., for reasons of toxicity) prior to end of cycle 8 and in the absence of withdrawal criteria, patients may continue to receive olaratumab monotherapy. For patients who discontinue olaratumab during the first 8 cycles of treatment, doxorubicin may be continued (for up to 8 cycles), provided no withdrawal criteria are met.

Doxorubicin Arm:

67 patients randomized in the Doxorubicin Arm receive 75 mg/m² doxorubicin on Day 1 of each cycle, for up to 8 cycles at the investigator's discretion. The actual (not planned) administration of each doxorubicin infusion defines Day 1 of each 21-day treatment cycle. During cycles 5 through 8, 750 mg/m² dexrazoxane may also be administered on Day 1 of each cycle, (at the investigator's discretion) prior to doxorubicin on Day 1 of each 21-day cycle. If the dose of doxorubicin is reduced at any point in the Study, the dose of dexrazoxane is to be reduced accordingly, maintaining a dosage ratio of 10:1. For example, if doxorubicin is reduced to 60 mg/m², then dexrazoxane is to be reduced to 600 mg/m². Doxorubicin Arm patients who develop PD during the initial 8 cycles may cross over to receive olaratumab monotherapy at the same dosing regimen and duration similar to those patients from the Combination Arm who continue on olaratumab monotherapy. Furthermore, patients in the Doxorubicin Arm who are discontinuing Study therapy due to therapy-related toxicity or complete doxorubicin treatment and experience SD or better may stay on Study schedule and assessments until PD is documented and then receive olaratumab monotherapy. In the case that a Doxorubicin Arm patient is to receive olaratumab monotherapy, PD must meet the protocol criteria.

Patients are assessed for tumor response every 6 weeks. Treatment is to continue until PD, development of unacceptable toxicity, noncompliance or withdrawal of consent by the patient, or investigator decision. Approximately 133 patients (65 in each treatment arm) were enrolled in the Phase 2 stage of this Study.

### Analysis of Efficacy Data

As per the pre-defined Study criteria, the Study is considered complete when the final analysis for OS is performed (which includes survival data for up to 24 months [2 years] after the last patient has been treated with his/her first dose of Study medication). After this point, any patients who are deriving benefit on Study treatment may continue to receive Study treatment after Study completion, and this treatment extension is to be decided on an individual basis.

The pre-defined end of trial is defined as when two years after the last patient has been treated with his/her first dose of Study medication, and the last patient has discontinued Study treatment and completed the 30-day safety follow-up visit or all olaratumab-related adverse events (AEs) have been followed until resolved, stabilized, returned to baseline, or deemed irreversible, whichever is the latest.

For Phase 2 data, the analysis is scheduled after 110 PFS events are observed. The analysis evaluates PFS, OS and OR.

### Analysis of Efficacy Data of Phase 2 Patients

For patients crossing over to olaratumab monotherapy after PD on the Doxorubicin Arm, all efficacy endpoints except OS are summarized separately for the post-cross-over period.

Primary Efficacy Endpoint

PFS is defined as the time from randomization until the first radiographic documentation of objective progression, or death from any cause. Patients who die without a reported prior progression are considered to have progressed on the day of their death. Patients who did not progress or are lost to follow-up are censored at the day of their last radiographic tumor assessment. If no baseline or post-baseline radiologic assessment is available, the patient is censored at the date of randomization. If death or PD occurs after two or more consecutive missing radiographic visits, censoring occurs at the date of the last radiographic visit prior to the missed visits. The use of a new anticancer therapy prior to the occurrence of PD results in censoring at the date of last radiographic assessment prior to initiation of new therapy.

As a sensitivity analysis, the actual reported date of progression and death is used to define PFS regardless of missing visits, early discontinuation, or start of new therapies to avoid informative censoring. Additionally, symptomatic deterioration may also be added as a progressive event for another sensitivity analysis.

The Kaplan-Meier method is used to estimate the median PFS time, together with a 90% confidence interval (hereinafter “CI”). The 3-month PFS is also estimated. Comparison between arms is performed using the Log-rank test, and the hazard ratios (HR) are estimated by a Cox proportional hazards regression model. Only when there are a sufficient number of patients in each stratum, the stratified analysis may be performed. Otherwise, the stratification factors may be treated as covariates in the Cox model to estimate the HR and 90% confidence limit.

Secondary Efficacy Endpoints

Secondary efficacy endpoints include OS, Objective Response Rate (“ORR”), and the association between tumor PDGFRα expression and clinical outcomes, including PFS, ORR, etc. The analysis of the secondary endpoints may be adjusted for the stratification factors.

OS is defined as the time from the date of randomization to the date of death from any cause. If the patient is alive at the end of the follow-up period or is lost to follow-up, OS is censored on the last date the patient is known to be alive. OS is evaluated by the Kaplan-Meier method and a 90% CI is provided for the median OS. The HR and 90% confidence limit for OS are estimated from the Cox regression model, taking the stratification factors as covariates. As a sensitivity analysis, patients in the Doxorubicin Arm are censored at the date of cross-over, since the OS endpoint is confounded by cross-over.

The ORR is equal to the proportion of patients achieving a best overall response of PR or CR (PR+CR) according to RECIST (1.1), from the start of the treatment until PD/recurrence. The ORR in each treatment group is compared using Fisher's exact test. Exact confidence bounds (90% CI) are determined.

For patients in the Doxorubicin Arm who crossed over to olaratumab therapy, the best overall response is assessed separately for prior to and after the start of olaratumab therapy.

The duration of response for responders only is measured from the time measurement criteria are first met for CR/PR (whichever is first recorded) until the first date that the criteria for PD are met, or death. Duration of response is estimated with the Kaplan-Meier method; a 90% CI is provided for the median duration of response. Patients who do not relapse are censored at the day of their last objective tumor assessment.

### Phase 2 Analysis Results in ‘The Study’

PFS:

Final analysis of the Combination Arm and Doxorubicin Arm showed a median PFS of 6.6 months (90% CI=4.1-8.3; interquartile range [IQR], 2.7-10.2) and 4.1 months (95% CI, 2.8-5.4; IQR, 1.6-7.4) respectively. This improvement in favor of olaratumab plus doxorubicin met the protocol-defined significance level of 0.1999 for final progression-free survival (stratified HR, 0.672; 95% CI, 0.442-1.021; p=0.0615). A blinded independent retrospective review of the radiologic scans showed a comparable HR (0.670; 95% CI, 0.04-1.12; p=0.1208) and a median progression-free survival of 8.2 months (95% CI, 5.5-9.8; IQR, 3.0-11.6) for the Combination Arm and 4.4 months (95% CI, 3.1-7.4; IQR, 1.5-8.6) for the Doxorubicin Arm.

OS:

Final analysis of the Combination Arm and the Doxorubicin Arm showed a median OS of 26.5 months (95% CI, 20.9-31.7; IQR, 13.8 to not evaluable) and 14.7 months (95% CI, 9.2-17.1; IQR, 5.5-26.0), respectively. The stratified HR for this analysis (stratified HR, 0.46; 95% CI, 0.30-0.71; p=0.0003) was consistent across the subgroup stratification factors including histological tumour type (leiomysarcoma vs non-leiomyosarcoma), number of lines of previous treatment (0 vs≧1), and PDGFRα status.

ORR/Disease Control Rate:

The objective response rate was 18.2% (95% CI, 9.8-29.6) in the olaratumab plus doxorubicin arm, and 11.9% (95% CI, 5.3-22.2) in the doxorubicin arm (p=0.3421). The objective response rate for the independent assessment was 18.2% (95% CI, 29.6-29.8) in the olaratumab plus doxorubicin arm, and 7.5% (95% CI, 2.5-16.6) in the doxorubicin arm (p=0.0740).

From a statistical perspective, phase 2 trials are typically designed to illustrate PFS, not OS, in part, due to the limited sample size of the Study; phase 2 trials are typically not designed to demonstrate statistically significant evidence of an OS benefit and certainly not of the magnitude demonstrated in the Study. A relatively small population will not show statistical significance unless a remarkable difference is observed. It is unexpected for the data to show a clear statistical improvement in the OS in a phase 2 study; the clinical data from the Study, over the spectrum of subtypes, demonstrates a similar OS benefit trend.

In conclusion, this study of olaratumab in combination with doxorubicin met its predefined, statistical, primary endpoint for progression-free survival and achieved a highly statistically significant improvement of 11.8 months in median overall survival over doxorubicin alone. Importantly, the improvement in median overall survival was achieved without an increase in serious toxicity, despite a higher cumulative exposure to doxorubicin. When the PFS and OS of the Study are compared to the currently available and recently investigated treatments, significant improvements over the current standard of care in terms of (1) a lower hazard ratio and (2) a larger improvement in median survival time, illustrate a clear unexpected benefit.

Although, olaratumab demonstrated overall survival across all subtypes, not all patient populations saw this benefit. Further pharmacokinetics (PK) and exposure-response analysis explores the change in dosing strategy needed for this patient population.

### Pharmacokinetics, Exposure-Response, and Modelling Analysis

Pharmacokinetics and Exposure-response Analysis of The “Study”:

The PFS, OS, PK and safety data from patients who received olaratumab in the Study, as discussed above, were analyzed.

The effect of olaratumab serum levels is explored using average serum concentration (Cavg) and trough serum concentration after cycle 1 (Cₘᵢₙ₁). The PFS and OS data are analyzed using a matched case-control (MCC) analysis across quartiles of olaratumab serum levels and a time-to-event (survival) model with a constant baseline hazard and a Hill function to describe the effect of olaratumab. The rate of treatment-emergent adverse events (TEAEs) is compared across quartiles of olaratumab exposure.

Olaratumab mean maximum serum concentration (Cₘₐₓ) reach 284 μg/mL (geometric coefficient of variation in % [CV %], 23.3) and 293 μg/mL (CV %, 30.5) after the first and second doses and return to a mean trough serum concentration (Cₘᵢₙ) of 66.5 μg/mL (CV %, 40.4) at the end of the cycle. Steady state is reached during cycle 3; mean steady state Cₘₐₓ and Cₘᵢₙ ranged from 419 μg/mL (CV %, 26.2) through 487 μg/mL (CV %, 33.0) and from 123 μg/mL (CV %, 31.2) through 156 μg/mL (CV %, 38.0) across cycles 4 through 9. Individual apparent terminal elimination half-life estimates of 6.67 days and 14.4 days are obtained during cycle 3. Olaratumab serum levels observed in patients randomized to the doxorubicin group, who received olaratumab monotherapy after disease progression, were similar to those observed in patients in the olaratumab plus doxorubicin arm. Exposure-response analyses indicate that patients in the upper quartiles of olaratumab serum exposure showed a greater improvement in progression-free survival and overall survival, regardless of the pharmacokinetic endpoint considered (Cₘᵢₙ at the end of cycle 1, or average serum concentration throughout the treatment duration); refer to Table 1 and 2.

TABLE 1
Matched-Case Control Analysis of Overall Survival by
Cₘᵢₙ₁ and C_{avg} Quartiles

| | Number of | | | |
|---|---|---|---|---|
| | patients/ | | | |
| | Number of | | | |
| Olaratumab | events | | Hazard | |
| Exposure | Olara + | | Ratio | p-value |
| Quartiles (μg/mL) | Dox | Dox | (95% CI) | (Wald's) |

Quartiles based on Cₘᵢₙ₁

| | | | | |
|---|---|---|---|---|
| Q1 (12.3-<62.8) | 15/13 | 15/12 | 1.355 (0.617, 2.976) | 0.4495 |
| Q2 (≧62.8-<86.9) | 16/10 | 16/13 | 0.528 (0.231, 1.206) | 0.1299 |
| Q3 (≧86.9-<105.6) | 15/5 | 15/8 | 0.386 (0.126, 1.182) | 0.0955 |
| Q4 (≧105.6-188.1) | 16/9 | 16/10 | 0.812 (0.330, 2.001) | 0.6510 |

Quartiles based on C_{avg}

| | | | | |
|---|---|---|---|---|
| Q1 (56 <134.4) | 15/13 | 15/13 | 1.024 (0.474, 2.215) | 0.9509 |
| Q2 (≧134.4-<175.2) | 16/9 | 16/14 | 0.367 (0.158, 0.851) | 0.0195 |
| Q3 (≧175.2-<249.9) | 15/7 | 15/7 | 0.717 (0.251, 2.046) | 0.5345 |
| Q4 (≧249.9-347.3) | 16/8 | 16/11 | 0.561 (0.226, 1.397) | 0.2145 |

Abbreviations:
C_{avg} = average concentration over patient's entire treatment;
CI = confidence interval;
Cₘᵢₙ₁ = trough serum level at the end of the first cycle of treatment;
Dox = doxorubicin;
Olara = olaratumab;
Q = quartile.

TABLE 2
Matched-Case Control Analysis of Progression-Free Survival
by Cₘᵢₙ₁ and C_{avg} Quartiles

| | Number of | | | |
|---|---|---|---|---|
| | patients/ | | | |
| | Number of | | | |
| Olaratumab | events | | Hazard | |
| Exposure | Olara + | | Ratio | p-value |
| Quartiles (μg/mL) | Dox | Dox | (95% CI) | (Wald's) |

Quartiles based on Cₘᵢₙ₁

| | | | | |
|---|---|---|---|---|
| Q1 (12.3-<62.8) | 15/13 | 15/11 | 1.810 (0.801, 4.087) | 0.1534 |
| Q2 (≧62.8-<86.9) | 16/14 | 16/9 | 1.516 (0.654, 3.510) | 0.3318 |
| Q3 ((≧86.9-<105.6) | 15/13 | 15/10 | 0.446 (0.192, 1.033) | 0.0596 |
| Q4 ((≧105.6-188.1) | 16/14 | 16/11 | 0.856 (0.386, 1.899) | 0.7027 |

Quartiles based on C_{avg}

| | | | | |
|---|---|---|---|---|
| Q1 (56 <134.4) | 15/13 | 15/14 | 1.828 (0.846, 3.951) | 0.1249 |
| Q2 (≧134.4-<175.2) | 16/15 | 16/10 | 1.165 (0.514, 2.642) | 0.7141 |
| Q3 ((≧175.2-<249.9) | 15/12 | 15/8 | 1.008 (0.408, 2.490) | 0.9865 |
| Q4 ((≧249.9-347.3) | 16/14 | 16/12 | 0.460 (0.211, 1.005) | 0.0513 |

Abbreviations:
C_{avg} = average concentration over patient's entire treatment;
CI = confidence interval;
Cₘᵢₙ₁ = trough serum level at the end of the first cycle of treatment;
Dox = doxorubicin;
Olara = olaratumab;
Q = quartile.

The survival model developed for PFS indicates that 66 μg/mL is also close to the minimum serum level needed to observe an improvement in the HR for PFS. Consistent with these results, a matched case-control analysis of OS and PFS per exposure quartiles based on the trough olaratumab serum concentration at the end of Cycle 1 (C_{min,1}) suggests that patients in the lowest exposure quartile (C_{min,1}<63 μg/mL, N=15) tend to experience disease progression within the first 2 cycles of treatment and, unlike the other quartiles, did not show OS improvement.

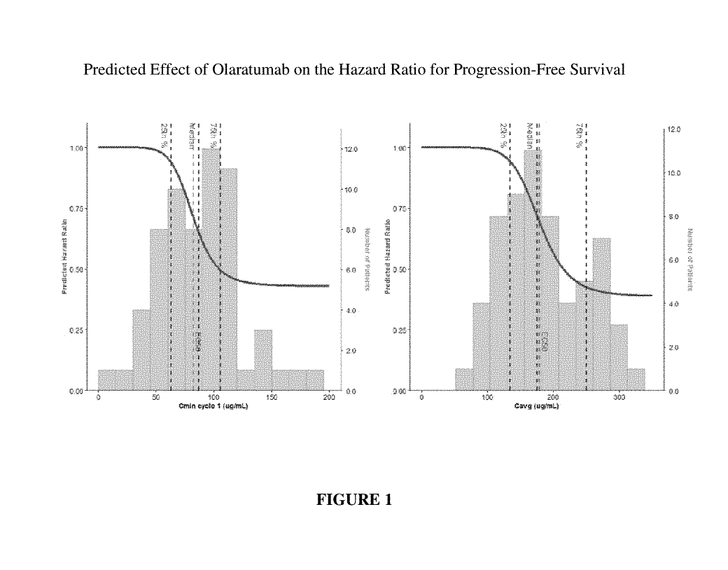

Further, FIG. 1, illustrates the predicted effect of olaratumab on HR for PFS. In FIG. 1, PFS as predicted by Cₘᵢₙ₁-based model is shown in the left panel and the C_{avg}-based model is shown in the right panel. The solid red lines describe the change in HR as a function of Cₘᵢₙ₁ and C_{avg}; the grey histograms describe the distribution of olaratumab Cₘᵢₙ₁ and C_{avg} in the Study experimental arm together with their quartiles (dashed blue lines); the green dashed lines indicate ECₘᵢₙ₁50 and EC_{avg}50. FIG. 1 shows that the HR for PFS is not predicted to change until the Cₘᵢₙ₁ or C_{avg} reaches values corresponding to the 25th percentile of their distribution in the study, which corresponds to the ECₘᵢₙ₁50 and EC_{avg}50 estimates for OS. Altogether, these predictions are consistent with the MCC analysis which shows that patients in the lowest exposure quartile tended to experience disease progression within the first 2 to 3 cycles of treatment and, unlike the other quartiles, did not have OS.

It is also important to note that in the higher quartiles of drug exposure in cycle 1 of the Study, there was no significant increase in observed toxicities (i.e. severe neutropenia or mucositis). This suggests that the earlier achievement (at the end of 1^{st} cycle) of therapeutic drug exposures due to using the loading dose may not be accompanied by increased toxicity—thus potential increased benefit to the patient without increased toxicity. See Tables 3 and 4.

TABLE 3
Treatment-Emergent Adverse Events Stratified by Olaratumab C_{min 1}
Quartile in The Study

| | | Olaratumab | | | | |
|---|---|---|---|---|---|---|
| | Doxorubicin | Overall | Q1 | Q2 | Q3 | Q4 |
| | n = 65 | n = 62 | n = 15 | n = 16 | n = 15 | n = 16 |
| C_{min 1} range | — | 12.3-188.1 | 12.3-<62.8 | 62.8-<86.9 | 86.9-<105.6 | 105.6-188.1 |
| (μg/mL) | | | | | | |
| Overall | | | | | | |
| Grade <= 2, % | 19 (29.2) | 12 (19.4) | 1 (6.7) | 5 (31.3) | 3 (20.0) | 3 (18.8) |
| Grade > 2, % | 45 (69.2) | 49 (79.0) | 13 (86.7) | 11 (68.8) | 12 (80.0) | 13 (81.3) |
| Grade >= 4, % | 20 (30.8) | 25 (40.3) | 7 (46.7) | 5 (31.3) | 7 (46.7) | 6 (37.5) |
| Neutropenia | | | | | | |
| Grade <= 2, %* | 3 (4.6) | 3 (4.8) | 0 | 2 (12.5) | 1 (6.7) | 0 |
| Grade > 2, %* | 22 (33.8) | 35 (56.5) | 9 (60.0) | 6 (37.5) | 11 (73.3) | 9 (56.3) |
| Grade >= 4, %* | 17 (26.2) | 23 (37.1) | 6 (40.0) | 5 (31.3) | 6 (40.0) | 6 (37.5) |
| Mucositis | | | | | | |
| Grade <= 2, %* | 20 (30.8) | 32 (51.6) | 8 (53.3) | 6 (37.5) | 9 (60.0) | 9 (56.3) |
| Grade > 2, %* | 3 (4.6) | 2 (3.2) | 0 | 2 (12.5) | 0 | 0 |
| Grade >= 4, %* | 0 | 0 | 0 | 0 | 0 | 0 |

Abbreviations:
C_{min 1} = trough serum concentration at the end of Cycle 1;
Q = quartile.
*consolidated treatment-emergent adverse event

TABLE 4
Treatment-Emergent Adverse Events Stratified by Olaratumab C_{avg}
Quartile in The Study

| | | Olaratumab | | | | |
|---|---|---|---|---|---|---|
| | Doxorubicin | Overall | Q1 | Q2 | Q3 | Q4 |
| | n = 65 | n = 62 | n = 15 | n = 16 | n = 15 | n = 16 |
| C_{avg} range | — | 56.0-347.3 | 56.0-<134.4 | 134.4-<175.2 | 175.2-<249.9 | 249.9-347.3 |
| (μg/mL) | | | | | | |
| Overall | | | | | | |
| Grade <= 2, % | 19 (29.2) | 12 (19.4) | 1 (6.7) | 5 (31.3) | 1 (6.7) | 5 (31.3) |
| Grade > 2, % | 45 (69.2) | 49 (79.0) | 13 (86.7) | 11 (68.8) | 14 (93.3) | 11 (68.8) |
| Grade >= 4, % | 20 (30.8) | 25 (40.3) | 6 (40.0) | 8 (50.0) | 6 (40.0) | 5 (31.3) |
| Neutropenia | | | | | | |
| Grade <= 2, %* | 3 (4.6) | 3 (4.8) | 0 | 2 (12.5) | 1 (6.7) | 0 |
| Grade > 2, %* | 22 (33.8) | 35 (56.5) | 9 (60.0) | 8 (50.0) | 11 (73.3) | 7 (43.8) |
| Grade >= 4, %* | 17 (26.2) | 23 (37.1) | 5 (33.3) | 7 (43.8) | 6 (40.0) | 5 (31.3) |
| Mucositis | | | | | | |
| Grade <= 2, %* | 20 (30.8) | 32 (51.6) | 6 (40.0) | 8 (50.0) | 8 (53.3) | 10 (62.5) |
| Grade > 2, %* | 3 (4.6) | 2 (3.2) | 0 | 2 (12.5) | 0 | 0 |
| Grade >= 4, %* | 0 | 0 | 0 | 0 | 0 | 0 |

Abbreviations:
C_{avg} = average concentration over patient's entire treatment;
Q = quartile.
*consolidated treatment-emergent adverse event

Population Pharmacokinetics Analysis of the Four Phase 2 Clinical Studies:

A population PK (PopPK) model was developed using PK data from 4 Phase 2 studies (www.clinicaltrials.gov study numbers NCT00918203 (A Randomized Phase 2 Study of Human Anti-PDGFRα Monoclonal Antibody (IMC-3G3) With Paclitaxel/Carboplatin or Paclitaxel/Carboplatin Alone in Previously Untreated Patients With Locally Advanced or Metastatic Non-Small Cell Lung Cancer), NCT01185964 (A Phase 1b/2 Randomized Phase 2 Study Evaluating the Efficacy of Doxorubicin With or Without a Human Anti-PDGFRα Monoclonal Antibody (IMC-3G3) in the Treatment of Advanced Soft Tissue Sarcoma), NCT00895180 (An Open Label, Phase 2 Study Evaluating the Safety and Efficacy of IMC-3G3 or IMC-1121B in Patients With Recurrent Glioblastoma Multiforme), and NCT01316263 (A Phase 2 Study of a Human Anti-PDGFRα Monoclonal Antibody (IMC-3G3) in Previously Treated Patients With Unresectable and/or Metastatic Gastrointestinal Stromal Tumors (GIST)). This model also indicates that steady-state olaratumab serum levels are not achieved until Cycle 3 irrespective of indication.

Conclusion:

Together, these findings suggest that clinical outcome for the lowest exposure quartile could be improved if patients were able to achieve therapeutic serum concentration levels (C_{min,1}≧63 μg/mL) of olaratumab earlier in treatment, before disease progression. These results highlight the complexity and difficulty involved in establishing dosing regimens that are able to achieve maximal benefit to all patient populations, without increased toxicity, particularly to patients with limited to no other beneficial treatment options currently available.

A Randomized, Double Blind, Placebo-Controlled, Phase 3 Trial of Doxorubicin Plus Olaratumab Versus Doxorubicin Plus Placebo in Patients with Advanced Soft Tissue Sarcoma (the “Phase 3 Study”)

The present invention is being further tested in the Phase 3 Study (trial number NCT02451943 available on clinicaltrials.gov https://clinicaltrials.gov/ct2/show/NCT02451943?term=ANNOUNCE&rank=1).

This Phase 3 Study consists of a 20 mg/kg dose of olaratumab administered on Days 1 and 8 of Cycle 1 followed by 15 mg/kg dose of olaratumab administered on Days 1 and 8 of every subsequent cycle. The Phase 3 Study Protocol is otherwise comparable to the design of the Study as set forth herein.

### CONCLUSIONS

In conclusion, based on the totality of work and trends seen in the aforementioned PK, exposure-response analysis, and modelling, the olaratumab dosing regimen of the present invention offers promise to a patient population with a clinically unmet need. The PK and exposure-response analyses suggest that this dosing strategy allows therapeutic olaratumab serum levels to be achieved as soon as the first cycle, and would minimize the number of patients whose Cₘᵢₙ₁ falls below 66 μg/mL at the start of treatment. The data also supports the assertion that toxicity is not increased (e.g. neutropenia) in spite of reaching therapeutic drug levels earlier, which maximizes the therapeutic benefit. Accordingly, use of the loading dose is likely to prevent a patient from encountering disease progression before they fall within the therapeutic window. Importantly, the use of 20 mg/kg during the first cycle is expected to yield maximum serum concentrations (Cₘₐₓ) that remain within the overall range observed in the Study (NCT01185964), for the previously unassisted patient populations as well as the patient populations that received the significant PFS and OS outcomes, which had an acceptable and monitorable safety profile. The present invention, currently being further explored in the Phase 3 Study, is expected to maximize the number of patients exposed to potentially therapeutic olaratumab serum levels, with minimal risk of increased toxicity, which can further optimize the benefit-risk ratio, and provide benefit for a patient population with a current unmet need.

Accordingly, the present invention provides an opportunity for improved patient outcomes similar to the Study (NCT01185964), for the previously unassisted patient populations as well as the patient populations that received the significant PFS and OS outcomes, while minimizing risk of increased toxicity, optimizes the benefit-risk ratio, and provide benefit for a patient population with a current unmet need.

We claim:

1. A method of administering an effective amount of olaratumab to a patient in need thereof comprising administering a loading dose of olaratumab followed by standard doses of olaratumab.

2. The method of claim 1 wherein the loading dose of olaratumab comprises 20 mg/kg of olaratumab.

3. The method of claim 2 wherein the 20 mg/kg of olaratumab is administered on day 1 and day 8 of a 21 day cycle.

4. The method of claim 2 wherein the 20 mg/kg of olaratumab is administered in the first cycle.

5. The method of claim 1 wherein the standard dose comprises 15 mg/kg of olaratumab.

6. The method of claim 5 wherein the 15 mg/kg of olaratumab is administered in the subsequent cycles.

7. The method of claim 5 wherein the 15 mg/kg of olaratumab is administered on day 1 and day 8 of a 21 day cycle.

8. The method of claim 1 wherein the patient has or is at risk of developing early progression.

9. The method of claim 1 wherein the patient has soft tissue sarcoma.

10. A method of administering an effective amount of olaratumab to a patient in need thereof comprising administering cycles of the olaratumab wherein a loading dose of 20 mg/kg of olaratumab is administered in the first cycle and a standard dose of 15 mg/kg of olaratumab is administered in the subsequent cycles.

11. The method of claim 10 wherein the loading dose of 20 mg/kg of olaratumab is administered in the first 21 day cycle and the standard dose of 15 mg/kg of olaratumab is administered in the subsequent 21 day cycles.

12. The method of claim 11 wherein the loading dose of 20 mg/kg of olaratumab is administered on day 1 and day 8 of the first 21 day cycle and the standard dose of 15 mg/kg of olaratumab is administered on day 1 and day 8 of the subsequent 21 day cycles.

13. The method of claim 10 wherein the patient has or is at risk of developing early progression.

14. The method of claim 10 wherein the patient has soft tissue sarcoma.

15. A therapeutic regimen for olaratumab in the treatment of cancer comprising 21 day cycles wherein a loading dose of 20 mg/kg of olaratumab is administered on day 1 and day 8 of the first 21 day cycle and 15 mg/kg of olaratumab is administered to a patient in need thereof on day 1 and day 8 of the subsequent 21 day cycles.

16. The therapeutic regimen of claim 15 wherein the patient has or is at risk of developing early progression.

17. The therapeutic regimen of claim 15 wherein the cancer is soft tissue sarcoma.

18. A method reducing the risk of disease progression of a patient having soft tissue sarcoma comprising administering a loading dose of olaratumab followed by standard doses of olaratumab.

19. The method of claim 18 wherein the loading dose of olaratumab comprises 20 mg/kg of olaratumab.

20. The method of claim 19 wherein the 20 mg/kg of olaratumab is administered on day 1 and day 8 of a 21 day cycle.

21. The method of claim 19 wherein the 20 mg/kg of olaratumab is administered in the first cycle.

22. The method of claim 18 wherein the standard dose comprises 15 mg/kg of olaratumab.

23. The method of claim 22 wherein the 15 mg/kg of olaratumab is administered in subsequent cycles.

24. The method of claim 22 wherein the 15 mg/kg of olaratumab is administered on day 1 and day 8 of a 21 day cycle.

25. A method of treating soft tissue sarcoma in a patient in need there of comprising administering a loading dose of olaratumab followed by standard doses of olaratumab.

26. The method of claim 25 wherein the loading dose of olaratumab comprises 20 mg/kg of olaratumab.

27. The method of claim 26 wherein the 20 mg/kg of olaratumab is administered on day 1 and day 8 of a 21 day cycle.

28. The method of claim 26 wherein the 20 mg/kg of olaratumab is administered in the first cycle.

29. The method of claim 25 wherein the standard dose comprises 15 mg/kg of olaratumab.

30. The method of claim 29 wherein the 15 mg/kg of olaratumab is administered in the subsequent cycles.

31. The method of claim 29 wherein the 15 mg/kg of olaratumab is administered on day 1 and day 8 of the 21 day cycle.

32. The method of claim 25 wherein the patient has or is at risk of developing early progression.
